(19) United States
(12) Patent Application Publication
(10) Pub. No.: US 2008/0070939 A1
(43) Pub. Date: Mar. 20, 2008

(54) LOCAL ANESTHETIC COMPOSITIONS CONTAINING DEXTROROTATORY MORPHINAN DERIVATIVES OR PHARMACEUTICALLY ACCEPTABLE SALTS THEREOF

(75) Inventors: JHI-JOUNG WANG, YUNG KANG CITY (TW); YU-WEN CHEN, YUNG KANG CITY (TW)

Correspondence Address: SHERIDAN ROSS PC, 1560 BROADWAY, SUITE 1200, DENVER, CO, 80202, US

(73) Assignee: CHI MEI FOUNDATION HOSPITAL, YUNG KANG CITY (TW)

(21) Appl. No.: 11/690,739

(22) Filed: Mar. 23, 2007

(30) Foreign Application Priority Data
Sep. 19, 2006 (TW) 095134596

(51) Int. Cl. A61K 31/439; A61P 23/00

(52) U.S. Cl. 514/289

(57) ABSTRACT

Dextrorotatory morphinan derivatives of formula (I) or pharmaceutically acceptable salts thereof were found to have local anesthetic effects:

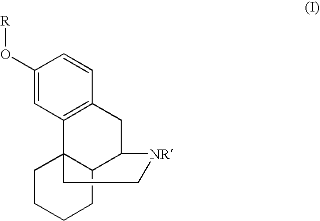

wherein R and R′ are independently selected from hydrogen and a methyl group, and at least one of R and R′ is a methyl group.

The dextrorotatory morphinan derivatives of formula (I) or pharmaceutically acceptable salts thereof can therefore be used in the manufacture of pharmaceutical compositions for local anesthesia that may provide a safe and prolonged local anesthetic effect.

### CROSS-REFERENCE TO RELATED APPLICATION

This application claims priority of Taiwan Application No. 095134596, filed on Sep. 19, 2006.

### BACKGROUND OF THE INVENTION

1. Field of the Invention

The invention relates to dextrorotatory morphinan derivatives or pharmaceutically acceptable salts thereof for use in local anesthesia. Specifically, this invention provides a pharmaceutical composition for local anesthesia, which comprises a dextrorotatory morphinan derivative or a pharmaceutically acceptable salt thereof, and a pharmaceutically acceptable carrier.

2. Description of the Related Art

Local anesthetics are drugs that can produce a reversible loss of sensation when applied to nerve tissues. They interfere with the conduction process of the nerve tissues by blocking use-dependent voltage-gated Na⁺ channels and thus inhibit initiation and propagation of action potentials of the nerve tissues (H. A. McLure and A. P. Rubin (2005), Minerva Anesthesiol., 71:59-74; A. Scholz (2002), Br. J. Anaesth., 89:52-61; H. A. Fozzard, et al. (2005), Curr. Pharm. Des., 11:2671-2686).

In 1860, Niemann first isolated an alkaloid, i.e., cocaine, from leaves of Erythroxylon coca Lam (Y. A. Ruetsch, et al. (2001), Curr. Top. Med. Chem., 1:175-182). In 1904, Einhorn synthesized the first procaine, a chemical derivative of cocaine (Y. A. Ruetsch, et al. (2001), Curr. Top. Med. Chem., 1:175-182). Thereafter, many local anesthetics were synthesized and extensively used in clinical practice. However, many of them were discarded due to some undesired side effects.

At present, more than ten types of local anesthetics are still in use, including, e.g., bupivacaine, lidocaine, cocaine, mepivacaine, tetracaine, and ropivacaine. These drugs can be cataloged into esters and amides according to their metabolic processes (H. A. McLure and A. P. Rubin (2005), Minerva Anesthesiol., 71:59-74; A. Scholz (2002), Br J. Anaesth., 89:52-61; H. A. Fozzard, et al. (2005), Curr. Pharm. Des., 11:2671-2686; Y. A. Ruetsch, et al. (2001), Curr. Top. Med. Chem., 1:175-182), in which the former type is metabolized mainly in the blood through hydrolysis by esterases whereas the latter type is metabolized in the liver. These two types of local anesthetics are very alike in some aspects. For example, they both have aromatic lipophilic and amine hydrophilic terminals in their chemical structures (H. A. McLure and A. P. Rubin (2005), Minerva Anesthesiol., 71:59-74). In terms of pharmacological mechanism, they both achieve effects of infiltrative cutaneous anesthesia, peripheral nerve blocking, and spinal/epidural anesthesia through Na⁺ channel blocking (H. A. Fozzard, et al. (2005), Curr. Pharm. Des., 11:2671-2686; H. A. McLure and A. P. Rubin (2005), Minerva Anesthesiol., 71:59-74; A. Scholz (2002), Br J. Anaesth., 89:52-61).

In addition, both of these two types of local anesthetics have poor systemic safety profiles of central nervous system toxicity and cardiovascular toxicity (H. A. Fozzard, et al. (2005), Curr. Pharm. Des., 11:2671-2686; Y. A. Ruetsch, et al. (2001), Curr. Top. Med. Chem., 1:175-182; L. E. Mather and D. H. Chang (2001), Drugs, 61:333-342). Particularly, during neural blockade, an accidental intravascular injection or an absolute overdose may result in convulsion or even cardiovascular collapse. Although some of them are emphasized to have lower toxicity to the central nervous system or the cardiovascular system, their differences in this aspect are actually minor. One possible explanation is that their chemical structures are similar.

Clinical use of these local anesthetics has continued for at least 140 years. It is about time that the medical sector endeavors to develop a new local anesthetic whose chemical structure is totally different from those of conventional local anesthetics for medical use.

Dextromethorphan is a dextrorotatory morphinan derivative whose chemical structure is similar to that of levorotatory morphinan derivative (e.g., levorphanol, codeine and morphine). Dextromethorphan was originally synthesized as a pharmacological alternative to morphine. Compared with levorotatory morphinan derivatives, dextromethorphan has little or even no opioid activity (A. A. Weinbroum, et al. (2000), Can. J. Anesth., 47:585-596), and it can effectively enhance the analgesic effect of opioid drugs and reduce the tolerance and dependence of morphine. In addition, dextromethorphan has an anticonvulsion property, and can protect the brain and spinal cord from neuronal damage induced by ischemia or excitatory amino acids (G. Trube and R. Netzer (1994), Epilepsia., 35:S62-S67).

For a long time, dextromethorphan has been extensively used in clinical practice as an antitussive, and is considered as a drug with a good margin of safety and a low incidence of side effects (J. L. Bem and R. Peck (1992), Drug Saf., 7:190-199; K. C. Carlsson, et al. (2005), Acta Anaesthesiol. Scand., 48:328-336; T. H. Duedahl, et al. (2006), Acta Anaesthesiol. Scand., 50:1-13; A. A. Weinbroum, et al. (2000), Can. J. Anesth., 47:585-596). In addition, dextromethorphan is also used in treating strokes, brain ischemia (J. A. Moses and D. W. Choi (1991), Stroke, 22:1075-1077; G. K. Steinberg, et al. (1993), Neurol. Res., 15:174-180), epilepsy (R. S. Fisher, et al. (1990), Neurology, 40:547-549), morphine dependence (S. D. Glick, et al. (2001), Eur. J. Pharmacol., 422:87-99; J. Mao, et al. (1996), Pain, 67:361-368), and acute pain and neuropathic pain (K. C. Carlsson, et al. (2005), Acta Anaesthesiol. Scand., 48:328-336; T. H. Duedahl, et al. (2006), Acta Anaesthesiol. Scand., 50:1-13; G. P. Joshi (2005), Int. Anesthesiol. Clin., 43:197-204; A. A. Weinbroum, et al. (2000), Can. J. Anesth., 47:585-596).

It is reported in literature that, in in vitro binding assays, dextromethorphan is an antagonist for N-methyl-D-asparate (NMDA) glutamate receptor (K. C. Carlsson, et al. (2005), Acta Anaesthesiol. Scand., 48:328-336; T. H. Duedahl, et al. (2006), Acta Anaesthesiol. Scand., 50:1-13; R. Netzer, et al. (1993), Eur. J. Pharmacol., 23:209-216) and nicotine/neuronal nicotinic receptor (M. I. Damaj, et al. (2005), J.P.E.T, 312:780-785), and has an effect of blocking voltage-gated Ca²⁺ and Na⁺ channels (R. Netzer, et al. (1993), Eur. J. Pharmacol., 23:209-216; G. Trube and R. Netzer (1994), Epilepsia., 35:S62-S67).

Previous studies show that dextromethorphan can be metabolized into dextrorphan through O-demethylation or into 3-methoxymorphinan through N-demethylation in the liver (S. Mordecai, et al. (1995), J. Clin. Psychopharmacol., 15:263-269). Thereafter, dextrorphan and 3-methoxymorphinan can be respectively metabolized into 3-hydroxymorphinan through N-demethylation and O-demethylation. Referring to scheme 1 shown hereinbelow, the chemical structures of these metabolites are very similar to that of dextromethorphan, and dextrorphan is the major metabolite.

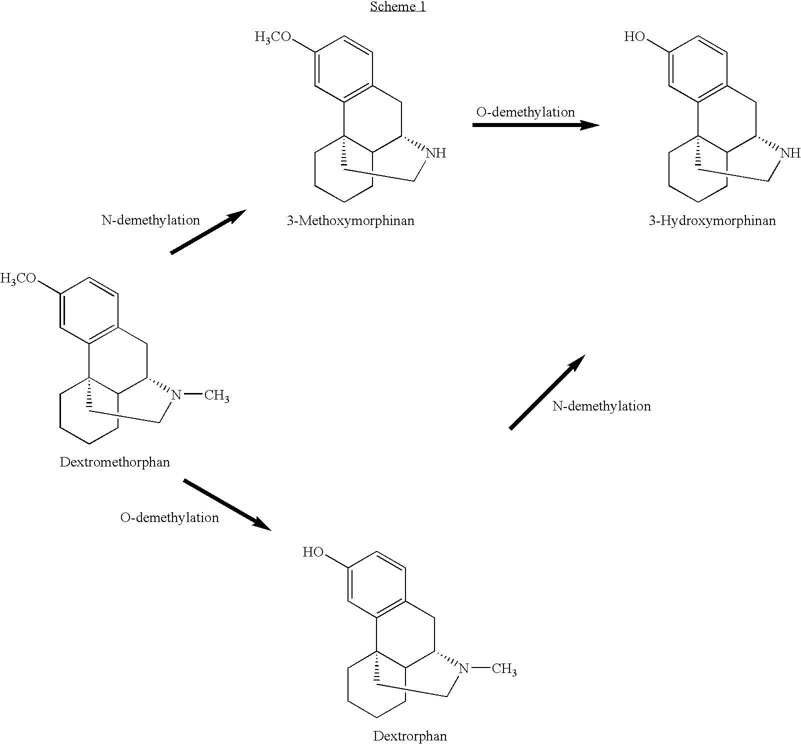

Like dextromethorphan, dextrorphan is also an antagonist for N-methyl-D-asparate (NMDA) glutamate receptor and nicotine/neuronal nicotinic receptor, and has a voltage-gated Ca²⁺ and Na⁺ channel blocking effect (M. I. Damaj, et al. (2005), J.P.E.T 312:780-785; G. Trube and R. Netzer (1994), Epilepsia., 35:S62-S67). In addition, dextrorphan is also effective in suppressing convulsion, protecting the brain and the spinal cord from damage caused by ischemia, and alleviating neuropathic pain (C. Du, et al. (1996), Brain Res., 718:233-236; H. Kato, et al. (1997), J. Thorac. Cardiovasc. Surg., 114:609-618; J. Mao, et al. (1993), Brain Res., 605: 164-168). Unlike dextromethorphan, dextrorphan can further bind to a phencyclidine receptor, and exert an effect similar to that of phencyclidine (J. I. Szekely, et al. (1991), Pharmacol. Biochem. Behav., 40:381-386).

It is mentioned in U.S. Pat. No. 5,352,683 granted to David J. Mayer et al. that when a chronic pain-alleviating amount of a non-toxic N-methyl-D-asparate receptor antagonist (such as dextromethorphan, dextrorphan, ketamine, or pharmacologically acceptable salts thereof) is administered to a mammal suffering from chronic pain, the chronic pain of the mammal can be alleviated. The non-toxic N-methyl-D-asparate receptor antagonist is administered independently or in combination with a local anesthetic, and is selectively in a sustained release dosage form.

In U.S. Pat. No. 5,502,058 granted to David J. Mayer et al., there is disclosed a method of alleviating pain (such as neuropathic pain or acute inflammatory pain). The method includes administering to a mammal that is either exhibiting pain or is about to be subjected to a pain-causing event, a pain alleviating/pain suppressing amount of at least one non-toxic antagonist for the N-methyl-D-aspartate receptor (e.g., dextrorphan), or a metabolic precursor of such antagonist (e.g., dextromethorphan), or at least one non-toxic substance that blocks a major intracellular consequence of N-methyl-D-aspartate receptor activation, e.g., a phenothiazine (such as trifluoperazine).

In U.S. Pat. No. 6,825,203 B2 granted to Gavril Pasternak and Yuri Kolesnikov, there is disclosed a topical pharmaceutical composition, which is formulated with at least one local anesthetic and at least one opioid analgesic in a topical excipient. Said patent also provides methods for relieving pain in a subject through topical administration of the pharmaceutical composition in an amount and a duration sufficient to synergistically potentiate an antinociceptive response. Synergistic potentiation of analgesia through topical administration of a local anesthetic/opioid pharmaceutical composition provides a new and improved approach to peripheral pain management.

Dextromethorphan and metabolites thereof are totally different from conventional local anesthetics in chemical structure, but have the same Na⁺ channel blocking effect as the conventional local anesthetics. According to the applicants' knowledge, no scientific documents or prior patent publications have ever disclosed use of dextromethorphan or the metabolites thereof on local anesthesia.

### SUMMARY OF THE INVENTION

Therefore, in a first aspect, this invention provides the use of a dextrorotatory morphinan derivative of the following formula (I) or a pharmaceutically acceptable salt thereof in the preparation of a pharmaceutical composition for local anesthesia:

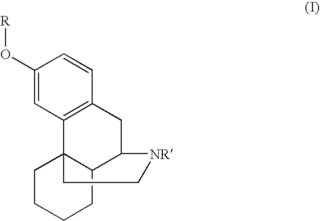

where R and R′ are independently selected from hydrogen and a methyl group, and at least one of R and R′ is a methyl group.

In a second aspect, this invention provides a pharmaceutical composition for local anesthesia. The composition includes a dextrorotatory morphinan derivative of formula (I) or a pharmaceutically acceptable salt thereof as described above, and a pharmaceutically acceptable carrier.

In a third aspect, this invention provides a method of applying local anesthesia to a subject (including human beings and animals). The method includes parenterally administering the aforesaid pharmaceutical composition to a subject in need of local anesthesia.

### BRIEF DESCRIPTION OF THE DRAWINGS

Other features and advantages of this invention will become apparent in the following detailed description of the preferred embodiment with reference to the accompanying drawings, of which:

FIG. 1 shows the spinal blockades on the motor function, proprioception and nociception produced in rats by bupivacaine hydrochloride, dextromethorphan hydrobromide monohydrate, dextrorphan tartrate, and 3-methoxymorphinan hydrochloride (each at an injection dose of ED₇₅) over time, the data being expressed in mean±SEM;

FIG. 2 shows the dose-response curves of bupivacaine hydrochloride, dextromethorphan hydrobromide hydrochloride, dextrorphan tartrate, and 3-methoxymorphinan hydrochloride on the spinal blockades of the motor function, proprioception and nociception produced in rats, the data being expressed in mean±SEM;

FIG. 3 shows the duration of bupivacaine hydrochloride, dextromethorphan hydrobromide hydrochloride, dextrorphan tartrate and 3-methoxymorphinan hydrochloride on the spinal blockades of the motor function, proprioception and nociception produced in rats at injection doses of ED₂₅, ED₅₀ and ED₇₅, the data being expressed in mean±SEM;

FIG. 4 shows the spinal blockades on the motor function, proprioception and nociception in rats as produced by dextromethorphan hydrobromide monohydrate when administered independently or when co-administered with bupivacaine hydrochloride over time (the data being expressed in mean±SEM; for each drug, the injection dosage was double the ED₅₀ when administered independently, and the dosage was ED₅₀ when co-administered);

FIG. 5 shows the spinal blockades on the motor function, proprioception and nociception produced in rats by dextrorphan tartrate when administered independently or when co-administered with bupivacaine hydrochloride over time (the data being expressed in mean±SEM; for each drug, the injection dosage was double the ED₅₀ when administered independently, and the dosage was ED₅₀ when co-administered);

FIG. 6 shows the spinal blockades on the motor function, proprioception and nociception produced in rats by 3-methoxymorphinan hydrochloride when administered independently or when co-administered with bupivacaine hydrochloride over time (the data being expressed in mean±SEM; for each drug, the injection dose was double the ED₅₀ when administered independently, and the dose was ED₅₀ when co-administered);

FIGS. 7A and 7B respectively show the sciatic nerve block on the motor function, proprioception and nociception produced in rats by dextromethorphan hydrobromide monohydrate and lidocaine hydrochloride over time when the dosage is 6.7 mg/kg (the data being expressed in mean±SEM);

FIG. 8 shows the dose-response curves of lidocaine hydrochloride, dextromethorphan hydrobromide monohydrate, dextrorphan tartrate, and 3-methoxymorphinan hydrochloride on the sciatic nerve block of the motor function, proprioception and nociception produced in rats (data being expressed in mean±SEM);

FIG. 9 shows the duration of lidocaine hydrochloride, dextromethorphan hydrobromide monohydrate, dextrorphan tartrate, and 3-methoxymorphinan hydrochloride on the sciatic nerve block of the motor function, proprioception and nociception produced in rats when the doses were ED₂₅, ED₅₀ and ED₇₅ (the data being expressed in mean±SEM);

FIG. 10 shows the sciatic nerve block on the motor function, proprioception and nociception produced in rats by dextromethorphan hydrobromide monohydrate when administered independently or when co-administered with lidocaine hydrochloride over time (the data being expressed in mean±SEM; for each drug, the injection dose was double the ED₅₀ when administered independently, and the dose was ED₅₀ when co-administered);

FIG. 11 shows the sciatic nerve block on the motor function, proprioception and nociception produced in rats by dextrorphan tartrate when administered independently or when co-administered with lidocaine hydrochloride over time (the data being expressed in mean±SEM; for each drug, the injection dose was double the ED₅₀ when administered independently, and the dose was ED₅₀ when co-administered);

FIG. 12 shows the sciatic nerve block on the motor function, proprioception and nociception produced in rats by 3-methoxymorphinan hydrochloride when administered independently or when co-administered with lidocaine hydrochloride over time (the data being expressed in mean±SEM; for each drug, the injection dose was double the ED₅₀ when administered independently, and the dose was ED₅₀ when co-administered);

FIG. 13 shows the inhibition of cutaneous trunci muscle reflex (CTMR) over time as produced in rats by subcutaneous injection of lidocaine hydrochloride, dextromethorphan hydrobromide monohydrate and dextrorphan tartrate at a dose of ED₇₅, the data being expressed in mean±SEM;

FIG. 14 shows the dose-response curves of lidocaine hydrochloride, dextromethorphan hydrobromide monohydrate and dextrorphan tartrate on cutaneous anesthesia produced in rats (the data being expressed in mean±SEM);

FIG. 15 shows the duration of lidocaine hydrochloride, dextromethorphan hydrobromide monohydrate, and dextrorphan tartrate on the cutaneous anesthesia produced in rats at doses of ED₂₅ ED₅₀ and ED₇₅, respectively, the data being expressed in mean±SEM;

FIGS. 16A and 16B respectively show the inhibition of cutaneous trunci muscle reflex (CTMR) produced in rats over time by dextromethorphan hydrobromide monohydrate and dextrorphan tartrate when administered independently or in combination with lidocaine hydrochloride (the data being expressed in mean±SEM; for each drug, the injection dose was double the ED₅₀ when administered independently, and the dose was ED₅₀ when co-administered); and

FIG. 17 shows the dose-mortality curves of lidocaine hydrochloride, dextromethorphan hydrobromide monohydrate and dextrorphan tartrate when administered intraperitoneally into the rats, the data being expressed in mean±SEM.

### DETAILED DESCRIPTION OF THE INVENTION

In developing drugs usable in local anesthesia, a dextrorotatory morphinan derivative of the following formula (I) or a pharmaceutically acceptable salt thereof was found to have potential industrial applicability. Therefore, this invention discloses the use of a dextrorotatory morphinan derivative of the following formula (I) or a pharmaceutically acceptable salt thereof in manufacture of a pharmaceutical composition for local anesthesia:

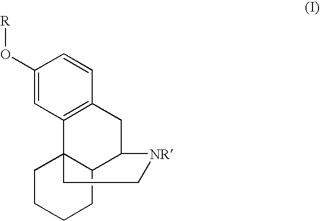

wherein R and R′ are independently selected from hydrogen and a methyl group, and at least one of R and R′ is a methyl group.

In a preferred embodiment of this invention, the dextrorotatory morphinan derivative of formula (I) is dextromethorphan.

In another preferred embodiment of this invention, the dextrorotatory morphinan derivative of formula (I) is 3-methoxymorphinan.

In still another preferred embodiment of this invention, the dextrorotatory morphinan derivative of formula (I) is dextrorphan.

The term “pharmaceutically acceptable salt” as used herein refers to a salt that can retain the biological effectiveness of free acids and bases of a designated compound and that is not a biologically undesirable salt. The pharmaceutically acceptable salt includes, but is not limited to, hydrochloride, hydrobromide, tartrate, maleate, oxalate, succinate, fumarate, sulfate, phosphate, acetate, propionate, caprate, caprylate, acrylate, formate, malonate, isobutyrate, caproate, enanthate, propargylate, sulfonate, citrate, lactate, and the like.

According to this invention, the pharmaceutically acceptable salt of dextrorotatory morphinan derivative of formula (I) can be a salt of inorganic acid (such as hydrochloric acid, hydrobromic acid, and sulfuric acid), a salt of organic acid (such as acetic acid, maleic acid, tartaric acid, succinic acid, citric acid, malic acid, oxalic acid, benzoic acid, methylsulfonic acid, and benzene sulfonic acid), and a salt of amino acid (such as arginine, aspartic acid, glutamic acid).

In a preferred embodiment of this invention, the dextrorotartory morphinan derivative of formula (I) or a pharmaceutically acceptable salt thereof is selected from the group consisting of dextromethorphan hydrobromide monohydrate, dextrorphan tartrate, and 3-methoxymorphinan hydrochloride.

According to this invention, the pharmaceutical composition is preferably administered parenterally. In a preferred embodiment of the invention, the pharmaceutical composition is administered via a route selected from the group consisting of epidural injection, intratracheal injection, subcutaneous injection, intrathecal injection, local transdermal delivery (e.g., through use of a skin patch or ointment), local transmembrane delivery (e.g., through use of a mucous membrane spray or gel), injections (e.g., peri-sciatic nerve injection) that result in a peripheral plexus block (such as a stellate ganglion block, a brachial plexus block, a solar plexus block, and a sciatic plexus block), and injections that result in a peripheral nerve block (such as an ulnar nerve block, a radial nerve block and a median nerve block). More preferably, the pharmaceutical composition is suitable for administration by subcutaneous injection, peri-sciatic nerve injection or epidural injection.

According to this invention, the pharmaceutical composition can be administered independently or in combination with an additional local anesthetic. Additional local anesthetics suitable for this invention can be selected from the group consisting of the following: bupivacaine, cocaine, mepivacaine, tetracaine, lidocaine, ropivacaine mesylate, or their pharmaceutically acceptable salts, and combinations thereof. Preferably, the additional local anesthetic is selected from the group consisting of bupivacaine, lidocaine or their pharmaceutically acceptable salts, and combinations thereof. More preferably, the additional local anesthetic is bupivacaine-hydrochloride or lidocaine-hydrochloride.

This invention also provides a pharmaceutical composition for local anesthesia, comprising:
(a) a dextrorotatory morphinan derivative of the following formula (I) or a pharmaceutically acceptable salt thereof:

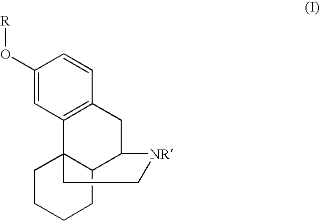

wherein R and R′ are respectively selected from hydrogen and a methyl group, and at least one of R and R′ is a methyl group; and
(b) a pharmaceutically acceptable carrier.

The above technical descriptions in connection with the use of the dextrorotatory morphinan derivative or a pharmaceutically acceptable salt thereof in local anesthesia according to this invention are also applicable to the pharmaceutical composition of this invention in this aspect.

According to this invention, the pharmaceutically acceptable carrier includes, but is not limited to, water, saline, phosphate buffered saline, sugar-containing solutions, alcohol-containing aqueous solutions (such as ethanol, propylene-glycol, glycerol, and mannitol), oils (such as peanut oil, olive oil, sesame oil, castor oil, cottonseed oil, and soybean oil), glycerine, organic solvents, and liposomes. Preferably, the carrier is 0.9% saline or 5% dextrose solution.

According to this invention, the pharmaceutical composition may further include an incipient selected from the group consisting of the following: stabilizers, chelating agents, preservatives, emulsifiers, suspending agents, diluents, and gelling agents.

The pharmaceutical composition according to this invention has been proven to have effects of infiltrative cutaneous anesthesia, peripheral nerve blocking, and spinal/epidural anesthesia, and is capable of providing a safe and prolonged local anesthetic effect to a subject in need of local anesthesia.

Therefore, this invention also provides a method of applying local anesthesia to a subject (including human beings and animals), the method including parenterally administering a pharmaceutical composition as described hereinabove to a subject in need of local anesthesia.

This invention also demonstrates that co-administration of dextromethorphan hydrobromide monohydrate or 3-methoxymorphinan hydrochloride with bupivacaine-hydrochloride produces an additive effect, whereas co-administration of dextrorphan tartrate with bupivacaine hydrochloride produces a synergistic effect. In addition, co-administration of dextromethorphan hydrobromide monohydrate, dextrorphan tartrate or 3-methoxymorphinan hydrochloride with lidocaine-hydrochloride produces an additive effect on sciatic nerve block. Furthermore, co-administration of dextromethorphan hydrobromide monohydrate or dextrorphan tartrate with lidocaine-hydrochloride will produce an additive effect of cutaneous anesthesia.

Therefore, according to the local anesthesia method of this invention, the pharmaceutical composition may be administered in combination with an additional local anesthetic to the subject. According to this invention, the pharmaceutical composition and the additional local anesthetic may be administered independently or together. According to this invention, the pharmaceutical composition and the additional local anesthetic may be administered to the subject at the same spot or at different spots. In a preferred embodiment of this invention, the pharmaceutical composition and the additional local anesthetic are simultaneously administered to the subject at the same spot. In another preferred embodiment of this invention, the pharmaceutical composition and the additional local anesthetic are simultaneously administered to the subject at different spots. Thus, according to this invention, the pharmaceutical composition can demonstrate a higher local anesthetic effect when co-administered with the additional local anesthetic than when the pharmaceutical composition is administered independently.

This invention will now be described in more detail with reference to the following examples, which are given for the purpose of illustration only and are not intended to limit the scope of this invention.

### EXAMPLES

### 1. Experimental Animals:

Male Sprague-Dawley rats (weighing about 200-350 g) obtained from the National Applied Research Laboratories National Laboratory Animal Center (Taiwan) were used in the following animal experiments. All the experimental animals were kept in a climate controlled room, and were allowed to obtain food and water freely during the entire experimental process. The climate controlled room was maintained at a temperature of 21° C. with a relative humidity of about 50%. Light was on a 12-hour light cycle (light on at 6:00 a.m.).

Before the experiment, the animals were given a week to be familiarized with the experimental environment and the experimental procedures so as to minimize stress-induced analgesia and advance the progress of the experiment. The animal experimental protocol was approved by the animal investigation committee of the Chi-Mei Medical Center, Tainan, Taiwan, and conformed to the recommendations and policies of the International Association for the Study of Pain.

### 2. Drugs:

Dextromethorphan hydrobromide monohydrate, dextrorphan tartrate, 3-methoxymorphinan hydrochloride, lidocaine-hydrochloride, and bupivacaine-hydrochloride were purchased from Sigma Chemical Co. (St. Louis, Mo.). All the drugs were freshly prepared in 5% dextrose solution or 0.9% saline before injection to form solutions having a pH falling within the range from 5.3 to 5.8. The solutions would be quickly buffered by the body fluids (pH 7.4) of the animals.

### Example 1

### Evaluation of Spinal Anesthetic Effects of dextromethorphan hydrobromide monohydrate, dextrorphan tartrate, 3-methoxymorphinan hydrochloride, and bupivacaine-hydrochloride on Rats

### Experimental Animals:

Male Sprague-Dawley rats (weighing about 300 to 350 g); for each dose of each drug, n=6.

### Drugs:

Dextromethorphan hydrobromide monohydrate, dextrorphan tartrate, 3-methoxymorphinan hydrochloride, and bupivacaine-hydrochloride were freshly prepared in 5% dextrose solution before intrathecal injection. The injection doses of each drug are as follows:
(1) Dextromethorphan hydrobromide monohydrate: 0.22 mg, 0.28 mg, 0.37 mg, 0.56 mg, 0.63 mg, 0.74 mg, 0.93 mg, and 1.11 mg;
(2) Dextrorphan tartrate: 0.15 mg, 0.20 mg, 0.25 mg, 0.31 mg, 0.41 mg, 0.61 mg, and 0.82 mg;
(3) 3-methoxymorphinan hydrochloride: 0.19 mg, 0.22 mg, 0.30 mg, 0.37 mg, 0.44 mg, 0.59 mg, 0.73 mg, and 0.88 mg; and
(4) Bupivacaine-hydrochloride: 0.04 mg, 0.06 mg, 0.08 mg, 0.10 mg, 0.12 mg, 0.16 mg, 0.24 mg, and 0.28 mg.

### Experimental Procedures:

### A. Drug Injection:

For intrathecal injection of the drugs, reference was made to the method described by Y. W. Chen, et al. in Pain (2004), 12:106-112. In brief, for preparation, the hairs on the back of each rat were shaved, and the shaved back was sterilized with iodine. Then, 100 μL of 1% lidocaine-hydrochloride was injected subcutaneously into each rat in the lumbar 4-5 intervertebral space. Next, 50 μL of 1% of lidocaine-hydrochloride was injected intramuscularly into each rat 0.5 cm to the left and right sides of a midpoint of a longitudinal line of the lumbar 4-5 intervertebral space and 0.5 cm deep.

After three minutes, 50 μL of each of the tested drugs was injected via a 27-gauge needle attached to a 100-μL syringe (Hamilton, Reno, Nev.) into the intrathecal space between the fourth and fifth lumbar vertebrae. The development of spinal blockade was observed from the paralysis of the hind limbs of the rats. Rats which showed unilateral blockade were excluded from the test, and were sacrificed using an overdose of ether.

### B. Neurobehavioral Evaluation:

Three neurobehavioral examinations, including motor function, proprioception, and nociceptive reaction, were conducted before intrathecal injection of the drugs and at 1, 5, 10, 20, 30, 40, 50, 60, 75, 90, 105, and 120 minutes after injection until the rats fully recovered.

The magnitude of nerve block (including blocking of motor function, proprioception, and nociceptive reaction) was expressed as the percentage of possible effect (hereinafter referred to as % PE), and the maximal value of % PE was expressed as the percentage of maximal possible effect (hereinafter referred to as % MPE).

Regarding the evaluation of motor function, proprioception and nociceptive reaction, reference was made to the methods described by, e.g., J. G. Thalhammer, et al., in Anesthesiology (1995), 82:1013-1025; Y. W. Chen, et al., in Pain (2004), 112:106-112; and P. Gerner, et al., in Anesthesiology (2000), 92:1350-1360, which are discussed briefly as follows:

### 1. Motor Function:

Motor function was evaluated by measuring the extensor postural thrust of the right hind limbs of the rats using a digital platform balance (Mettler Toledo, Switzerland, model PB1502-S). To test the extensor postural thrust, the rat was held upright with the hind limb extended, so that the body weight of the rat was supported by the distal metatarsus and toes. The extensor postural thrust could be measured as the force of the right hind limb of the rat thrusting the platform balance.

The control value (falling within the range from 1600 g to 1650 g) measured before injection of the drugs was considered as 0% motor block or 0% MPE. A reduction in the force, resulting in extensor muscle tone, was considered as a motor deficit. A force less than 20 g (i.e., a weight of the “flaccid limb”) was considered absence of an extensor postural thrust or 100% motor block or 100% MPE.

### 2. Proprioception:

Proprioception was evaluated based on resting posture and postural reactions (including tactile placing and hopping). First, the front half of the rat body and one of the hind limbs were lifted off the ground at the same time so that the rat was standing on one hind limb. Then, the rat was moved laterally, which normally evoked a prompt hopping response of the weight-bearing hind limb of the rat in the direction of movement so as to avoid falling. A predominantly proprioceptive block caused a delayed hopping followed by greater lateral hops. When proprioception was fully blocked, the rat would have no hopping maneuvers. Functional deficits were classified into four grades: 3 (normal or 0% MPE), 2 (slightly impaired), 1 (severely impaired), and 0 (completely impaired or 100% MPE).

### 3. Nociceptive Reaction:

Nociceptive reaction was evaluated by the withdrawal reflex or vocalization elicited by the pinch of a skin fold over the back at 1 cm near the proximal part of the tail, the lateral metatarsus of the bilateral hind limbs, and the dorsal part of the mid-tail. At each testing time, only one pinch was given to each of the abovementioned four testing sites, and the time interval between stimulations at different sites was around 2 seconds. Nociceptive reactions were classified into five grades: 4 (normal or 0% MPE), 3 (25% MPE), 2 (50% MPE), 1 (75% MPE), and 0 (absent or 100% MPE).

### C. Potency Evaluation:

According to the results obtained in the preceding section “B. Neurobehavioral evaluation,” dose-response curves were obtained for each drug by plotting the dose of each drug and the corresponding % MPE. These curves were fitted using a computer-derived SAS NLIN analysis (SAS Institute Inc., North Carolina), and the effective doses (ED₂₅, ED₅₀ and ED₇₅) of each drug which would cause 25%, 50% and 75% blockades were calculated. The potency of each drug on spinal blockades was compared using ED₅₀.

### D. Evaluation of Duration:

Duration was defined as the interval from injection of the tested drugs to complete recovery. The ED₂₅, ED₅₀ and ED₇₅ obtained according to the SAS NLIN analysis in the preceding section “C. Potency evaluation” were used in this experiment.

Intrathecal injections of ED₂₅, ED₅₀ and ED₇₅ of each drug were performed according to the method described in the preceding section “A. Drug injection,” and tests of motor function, proprioception and nociception were performed according to the method described in the preceding section “B. Neurobehavioral evaluation.” On an equi-potent basis (i.e., at the same effective doses), the duration of spinal blockade of each drug on motor function, proprioception, and nociception was measured independently and compared.

### E. Evaluation of Effect of Co-Administration:

The effect of co-administration of dextrorotatory hydrobromide monohydrate, dextrorphan tartrate, or 3-methoxymorphinan hydrochloride with bupivacaine hydrochloride on spinal blockade was also evaluated.

Regarding the method of evaluating the effect of co-administration, dextromethorphan hydrobromide monohydrate was used as an example. The rats were randomly divided into 3 groups (n=6 in each group), and received intrathecal injections of dextromethorphan hydrobromide monohydrate (dose: 2 times ED₅₀), bupivacaine hydrochloride (dose: 2 times ED₅₀), and a combination of dextromethorphan hydrobromide monohydrate and bupivacaine hydrochloride (dose of each drug: ED₅₀), respectively, according to the method described in the preceding section “A. Drug injection.” Thereafter, the motor function, proprioception and nociceptive reaction were evaluated according to the method described in the preceding section “B. Neurobehavioral evaluation.”

In addition, the effect of co-administration of dextrorphan tartrate or 3-methoxymorphinan hydrochloride was evaluated with reference to the above-described method.

After the tests, the time of measurement of each drug and its corresponding % PE were plotted to obtain the time-% PE curves of each drug. The area under the curve (AUC) of time-% PE was calculated using the trapezoidal rule, and was used to evaluate the effect of the co-administration of the drugs. Regarding the method of calculating AUC, reference can be made to, e.g., W. A. Ritschel and G. L. Kearns (2004), Handbook of basic pharmacokinetics—Including clinical applications., 6th edit. Am Pharm Assoc., pp. 179-187.

### F. Statistical Analysis:

Statistical software, SPSS for Windows® (version 10.0.7) was used. Data were represented as mean±SEM. Differences in ED₅₀ among the drugs were evaluated by a one-way analysis of variance (ANOVA), followed by a pairwise Tukey honest significant difference test (hereinafter referred to as the pairwise Tukey HSD test). Differences in duration among the drugs were evaluated by a two-way ANOVA, followed by the pairwise Tukey HSD test. Differences in AUC among different medications were evaluated by a one-way ANOVA, followed by the pairwise Tukey HSD test. If the result of statistical comparison p thus obtained was less than 0.05, this represents statistical significance.

### Result:

### 1. Time Courses of Spinal Blockades at ED₇₅:

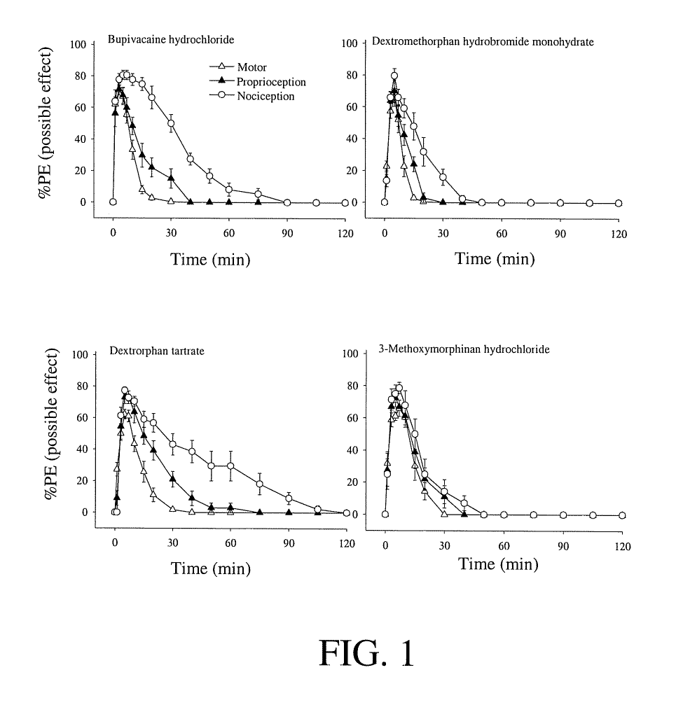

The time courses of spinal blockades of motor function, proprioception and nociceptive reaction at different doses of drugs were conducted. Due to the similarities of the graphs plotted for different doses, only those obtained at ED₇₅ were shown (see FIG. 1). As shown in FIG. 1, at ED₇₅, bupivacaine hydrochloride (ED₇₅ was 0.14 mg) produced about 71±3%, 72±4% and 81±3% MPE of spinal blockade of motor function, proprioception and nociceptive reaction with duration of about 25±3, 32±4 and 63±7 minutes, respectively. In addition, dextromethorphan hydrobromide monohydrate (ED₇₅ is 0.65 mg), dextrorphan tartrate (ED₇₅ was 0.45 mg) and 3-methoxymorphinan hydrochloride (ED₇₅ was 0.53 mg) produced about 66-80%, 62-77% and 66-79% MPE of spinal blockades of motor function, proprioception and nociceptive reaction, respectively, with duration of about 22-40, 30-65 and 28-35 minutes, respectively.

### 2. Potency Evaluation:

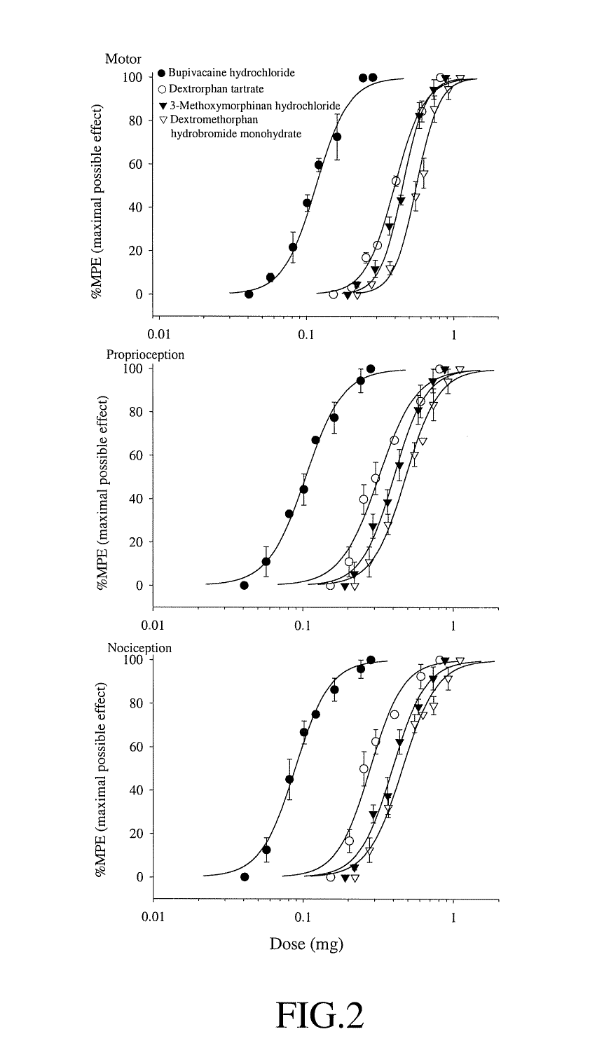

FIG. 2 shows the percentage of maximal possible effect (% MPE) of the drugs on spinal blockades at different doses. It can be seen from FIG. 2 that all the tested drugs produced dose-related spinal blockades of motor function, proprioception and nociceptive reaction. Table 1 shows the effective doses of the drugs on spinal blockades. It can be seen from Table 1 that on an ED₅₀ basis, the ranks of potencies of the drugs are: bupivacaine hydrochloride>dextrorphan tartrate>3-methoxymorphinan hydrochloride>dextromethorphan hydrobromide monohydrate (p<0.01).

TABLE 1
Effective doses of drugs on spinal blockades of motor function,
proprioception and nociceptive reaction in rats

| | | | Nociceptive | | | |
|---|---|---|---|---|---|---|
| | Motor function | Proprioception | reaction | Mean value | | |
| Drug | ED₅₀ (95% CI) | ED₅₀ (95% CI) | ED₅₀ (95% CI) | ED₂₅ | ED₅₀ | ED₇₅ |
| Bupivacaine | 0.12 (0.11–0.12) | 0.10 (0.10–0.11) | 0.09 (0.08–0.09) | 0.08 | 0.11 | 0.14 |
| hydrochloride (B) | | | | | | |
| Dextrorphan tartrate | 0.40 (0.39–0.42) | 0.32 (0.30–0.35) | 0.28 (0.26–0.30) | 0.25 | 0.33 | 0.45 |
| (DX) | | | | | | |
| 3-methoxymorphinan | 0.45 (0.43–0.47) | 0.40 (0.38–0.43) | 0.39 (0.37–0.42) | 0.33 | 0.41 | 0.53 |
| hydrochloride(3MM) | | | | | | |
| Dextromethorphan | 0.56 (0.54–0.59) | 0.48 (0.45–0.52) | 0.46 (0.43–0.49) | 0.39 | 0.50 | 0.65 |
| hydrobromide | | | | | | |
| monohydrate (DM) | | | | | | |

Note 1:
95% CI refers to 95% confidence interval.
Note 2:
Differences among the drugs were compared using a one-way analysis of variance followed by pairwise Tukey honest significant difference test.

### 3. Duration:

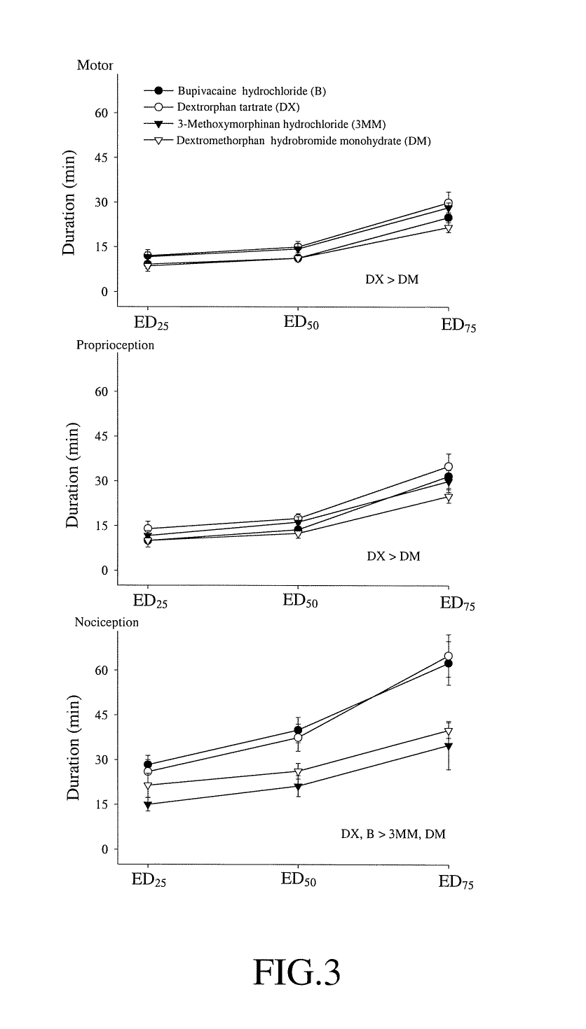

Duration of the drugs on spinal blockades of motor function, proprioception and nociceptive reaction were also evaluated. It can be seen from FIG. 3 that at the same effective doses (i.e., ED₂₅, ED₅₀ or ED₇₅), dextromethorphan hydrobromide monohydrate and 3-methoxymorphinan hydrochloride had similar reaction duration, whereas dextrorphan tartrate and bupivacaine hydrochloride had similar reaction duration.

### 4. Effect of Co-Administration:

The effect of co-administration of dextromethorphan hydrobromide monohydrate, dextrorphan tartrate or 3-methoxymorphinan hydrochloride with bupivacaine hydrochloride on spinal blockade was evaluated.

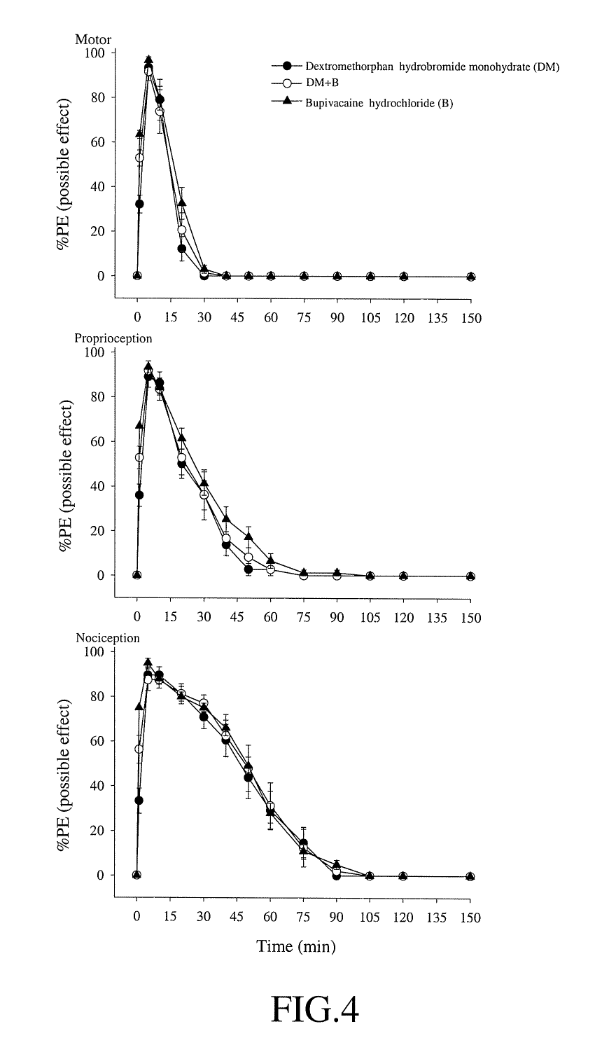

The results show that the AUC produced by the co-administration of dextromethorphan hydrobromide monohydrate and bupivacaine hydrochloride on the spinal blockade of motor function, proprioception and nociceptive reaction was between the AUCs (see Table 2 and FIG. 4) produced when dextromethorphan hydrobromide monohydrate and bupivacaine hydrochloride were administered independently.

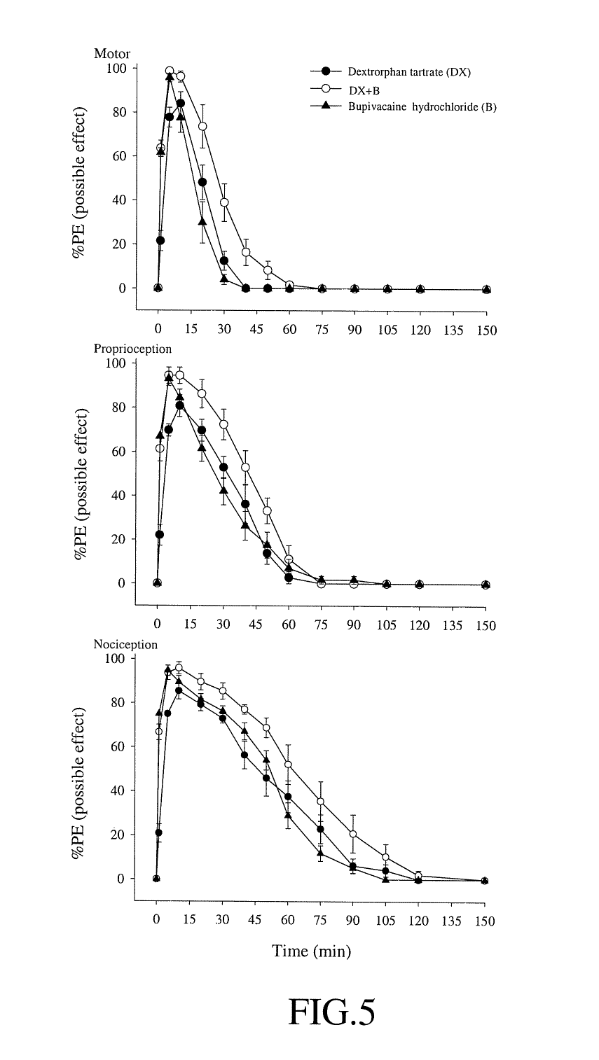

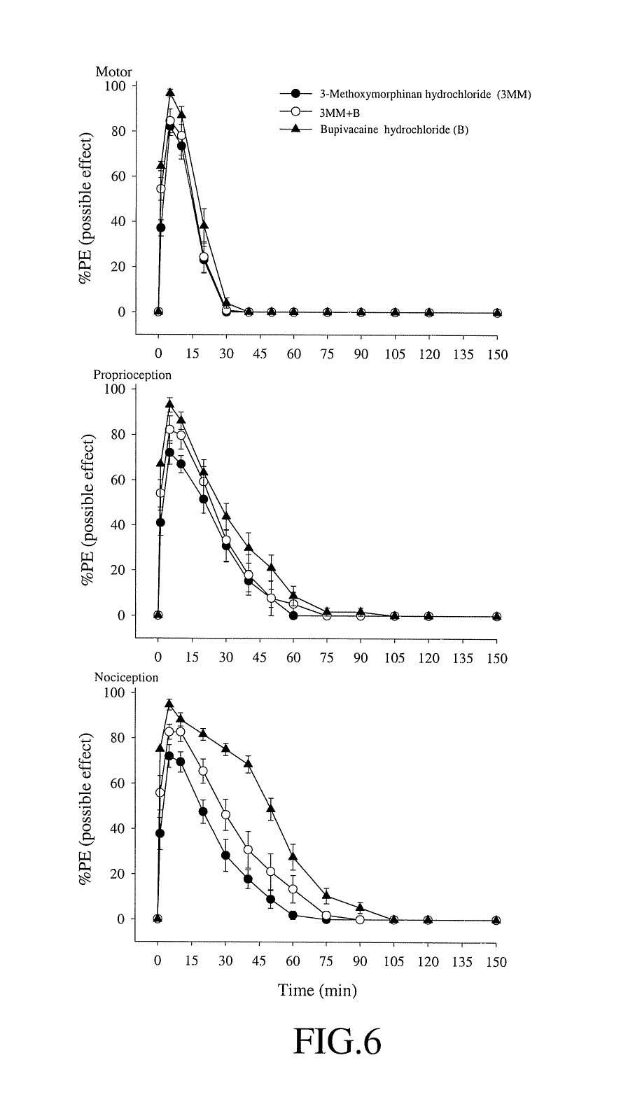

On the contrary, the AUC produced by the co-administration of dextrorphan tartrate and bupivacaine hydrochloride on the spinal blockade of motor function, proprioception and nociceptive reaction was greater than the AUCs (see Table 2 and FIG. 5) produced when dextrorphan tartrate and bupivacaine hydrochloride were administered independently. In addition, the co-administration of 3-methoxymorphinan hydrochloride and bupivacaine hydrochloride exhibited results similar to those obtained from the co-administration of dextromethorphan hydrobromide monohydrate and bupivacaine hydrochloride (see Table 2 and FIG. 6).

TABLE 2
Effect of co-administration of dextromethorphan hydrobromide monohydrate,
dextrorphan tartrate or 3-methoxymorphinan hydrochloride with bupivacaine
chloride on spinal blockade

| | Motor function | | proprioception | | Nociceptive reaction | |
|---|---|---|---|---|---|---|
| Dextromethorphan | 953 ± 114 | | 1985 ± 219 | | 4131 ± 385 | |
| hydrochloride monohydrate | | | | | | |
| (DM) | | | | | | |
| Dextromethorphan | 1073 ± 152 | | 2188 ± 348 | | 4445 ± 570 | |
| hydrobromide monohydrate + | | | | | | |
| bupivacaine (DM + B) | | | | | | |
| Bupivacaine hydrochloride (B) | 1292 ± 113 | | 2597 ± 245 | | 4378 ± 184 | |
| Dextrorphan tartrate (DX) | 1447 ± 163 | | 2532 ± 226 | | 4332 ± 382 | |
| Dextrorphan | 2663 ± 302 | DX + B > DX, B | 3660 ± 297 | DX + B > DX, B | 5922 ± 440 | DX + B > DX, B |
| tartrate + bupivacaine | | (p < 0.05) | | (p < 0.05) | | (p < 0.05) |
| hydrochloride (DX + B) | | | | | | |
| Bupivacaine hydrochloride | 1238 ± 147 | | 2638 ± 308 | | 4512 ± 229 | |
| (B) | | | | | | |
| 3-methoxymorphinan | 1265 ± 232 | | 1917 ± 254 | | 2081 ± 227 | |
| hydrochloride (3MM) | | | | | | |
| 3-methoxymorphinan | 1273 ± 185 | | 2683 ± 621 | | 3036 ± 345 | B > 3MM + B > 3MM |
| hydrochloride + bupivacaine | | | | | | (p < 0.05) |
| hydrochloride (3MM + B) | | | | | | |
| Bupivacaine hydrochloride (B) | 1383 ± 131 | | 2774 ± 299 | | 4410 ± 225 | |

Note 1:
Data were derived from AUC values in FIG. 4 to 6, and are expressed in mean ± SEM
Note 2: For each drug, the injection dosage was 2 ED₅₀ (i.e., 2 times ED₅₀) when administered independently, and the dosage was ED₅₀ when co-administered.
Note 3:
Differences among the drugs were compared using a one-way analysis of variance, followed by pairwise Tukey HSD test (p < 0.05).

From the aforesaid results, it has been demonstrated that all of dextromethorphan hydrobromide monohydrate, dextrorphan tartrate and 3-methoxymorphinan hydrochloride have a local anesthetic effect, of which dextrorphan tartrate exhibited the most potent effect, and the duration of the effect was the same as that of bupivacaine hydrochloride. In addition, co-administration of dextromethorphan hydrobromide monohydrate or 3-methoxymorhpinan hydrochloride with bupivacaine hydrochloride produced an additive effect on spinal blockade, whereas co-administration of dextrorphan tartrate and bupivacaine hydrochloride produced a synergistic effect.

### Example 2

### Evaluation of effects of dextromethorphan hydrobromide monohydrate, dextrorphan tartrate, 3-methoxymorphinan hydrochloride and lidocaine hydrochloride on Sciatic Nerve Blockade in Rats

### Experimental Animals:

Male Sprague-Dawley rats (weighing about 300 to 350 g); for each dose of each drug, n=6.

### Drugs:

Dextromethorphan hydrobromide monohydrate, dextrorphan tartrate, 3-methoxymorphinan hydrochloride, and lidocaine hydrochloride were freshly prepared in 0.9% saline before injection. The injection doses of each drug are as follows:
(1) Dextromethorphan hydrobromide monohydrate: 1.35 mg/kg, 2.04 mg/kg, 2.69 mg/kg, 4.04 mg/kg, 4.84 mg/kg, 5.38 mg/kg, 6.73 mg/kg, and 14 mg/kg;
(2) Dextrorphan tartrate: 5.93 mg/kg, 10.07 mg/kg, 14.51 mg/kg, 17.78 mg/kg, 20.76 mg/kg, 29.64 mg/kg, 32.62 mg/kg, and 70 mg/kg;
(3) 3-methoxymorphinan hydrochloride: 3.2 mg/kg, 4.25 mg/kg, 5.13 mg/kg, 6.29 mg/kg, 8.55 mg/kg, 12.84 mg/kg, and 25 mg/kg; and
(4) Lidocaine hydrochloride: 0.98 mg/kg, 1.56 mg/kg, 1.94 mg/kg, 2.55 mg/kg, 2.95 mg/kg, 3.16 mg/kg, and 6.7 mg/kg.

### Experimental Procedures:

### A. Drug Injection:

For the injection of drugs at the sciatic nerve notch, reference was made to the method described by P. Gerner, et al., in Anesthesiology (2000), 96: 1435-1442. In brief, the rats were slightly anesthetized by inhalation of a low concentration of sevoflurane. Then, 200 μL of tested drugs were injected at the sciatic nerve notch at the left hind limb of the rat using a 27-gauge needle connected to a tuberculin syringe (Becton Dickinson, USA, model BND309623-BX). Thereafter, development of sciatic nerve blockade was observed from the paralysis of the left hind limb of the rat. The right hind limb served as a control during the subsequent functional assay

### B. Neurobehavioral Evaluations

Evaluations of three neurobehavioral examinations, including motor function, proprioception and nociceptive reaction, were conducted 15 minutes before injection and 2, 5, 10, 20, 30, 40, 50, 60, 80, 100, 120 and 150 minutes after injection until complete recovery of the rats.

Evaluations of motor function, proprioception and nociceptive reaction were conducted based on the method described in Example 1 hereinabove The differences resided in that: For motor function, the extensor postural thrust was measured as the force of the left hind limb of the rat pushing against the platform of the scale, and a control value falling in a range from 240 g to 260 g was considered as 0% motor blockade or 0% MPE. For proprioception, the front half of the rat body and the right hind limb were off the ground at the same time so that the rat was standing on the left hind limb during the measurement. For nociceptive reaction, evaluation was based on the withdrawal reflex or vocalization elicited by pinching the fifth toe of the hind limb of the rat, and nociceptive reaction was divided into three grades: 2 (normal or 0% MPE), 1 (50% MPE), and 0 (absent or 100% MPE)(P. Gerner et al. (2002), Anesthesiology, 96:1435-1442 and Y. Sudoh, et al. (2003), Pain, 103:49-55).

### C. Potency Evaluation:

Potency evaluation was conducted based on the method described in Example 1.

### D. Evaluation of Duration:

Analysis of the duration was conducted with reference to the method described in Example 1. The differences resided in that: The sciatic nerve notch injections of ED₂₅, ED₅₀ and ED₇₅ were conducted based on the method described in the preceding section “A. Drug injection” of this example. Evaluations of motor function, proprioception and nociceptive reaction were conducted based on the method described in section “B. Neurobehavioral evaluations” of this example.

### E. Evaluation of Effect of Co-Administration:

Evaluations of the effect of co-administration were conducted with reference to the method described in Example 1. The differences resided in that: Bupivacaine hydrochloride was replaced by lidocaine hydrochloride; and the peri-sciatic nerve injection of the drugs, and the evaluations of motor function, proprioception and nociceptive reaction were conducted according to the methods described in the sections “A. Drug injection” and “B. Neurobehavioral evaluations” of this example.

### F. Statistical Analysis:

The statistical analysis was conducted based on the method described in Example 1. The differences resided in that: Before the statistical analysis, Shapiro-Wilk test was used to evaluate whether the data were, theoretically, normally distributed. After testing, it was found that the majority of the data fitted the normality assumption. Therefore, a parametric test was used in subsequent data analyses.

### Results:

### 1. Time Courses of Sciatic Nerve Blockades at a Dose of 6.7 mg/kg:

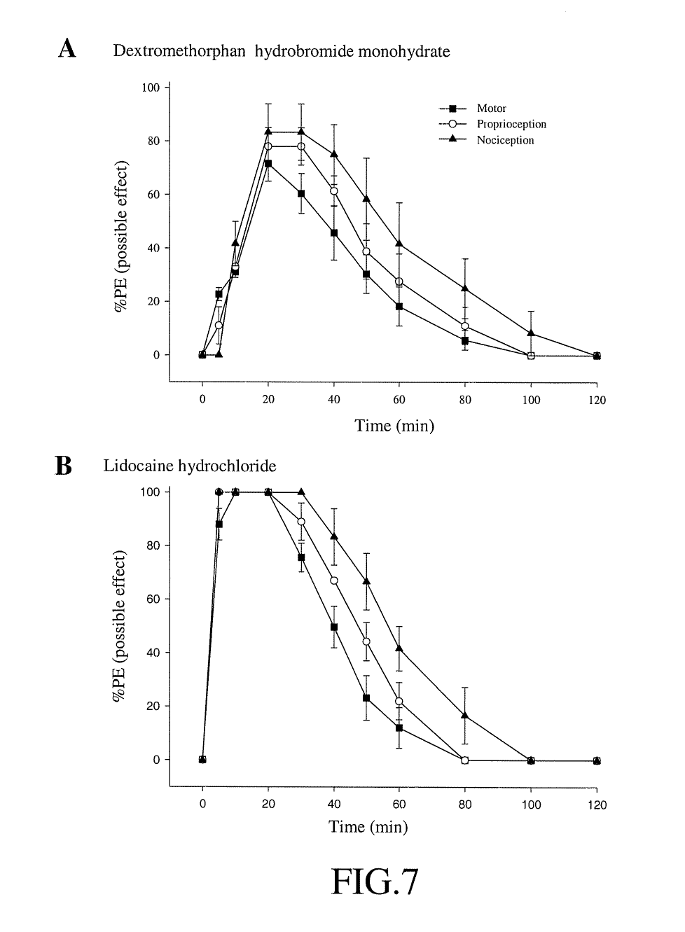

The time courses of sciatic nerve blockades of motor function, proprioception and nociceptive reaction at different doses of drugs were conducted. Due to the similarities of the graphs plotted for different doses, only those obtained when the injection dosage was 6.7 mg/kg were shown (see FIG. 7). The results show that, at a dosage of 6.7 mg/kg, dextromethorphan hydrochloride monohydrate produced about 71±7%, 78±7% and 83±11% MPE of sciatic nerve blockade of motor function, proprioception and nociceptive reaction with durations of about 77±10, 78±8 and 85±11 minutes, respectively (see FIG. 7A), whereas lidocaine produced 100% MPE of sciatic nerve blockade of motor function, proprioception and nociceptive reaction with durations of about 63±6, 73±4 and 83±6 minutes, respectively (see FIG. 7B).

### 2. Potency Evaluation:

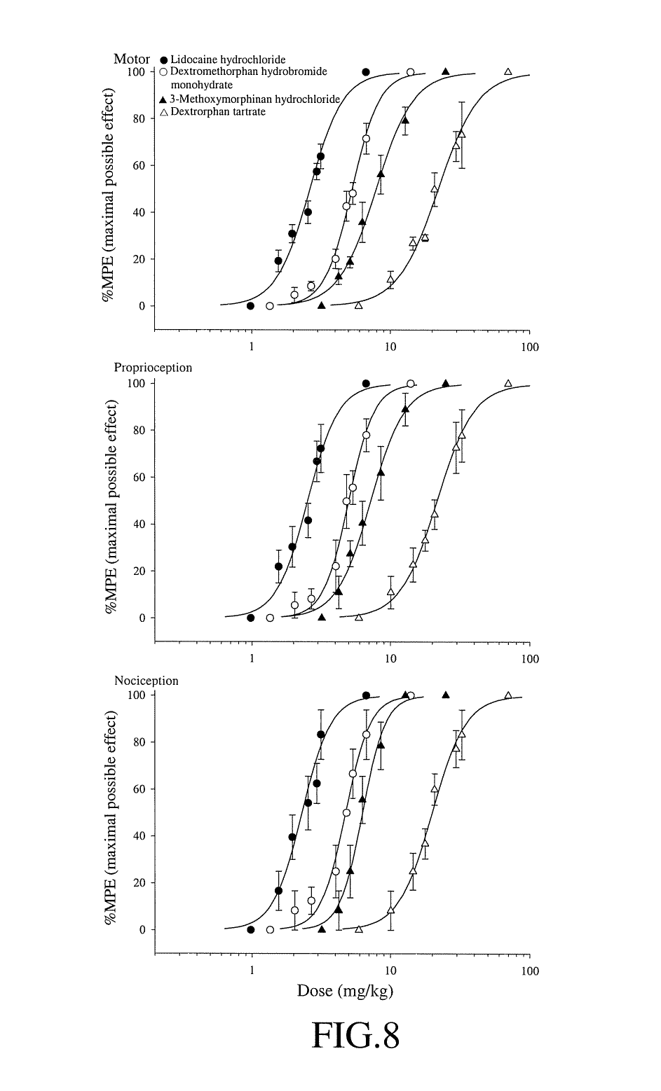

FIG. 8 shows the percentage of maximal possible effect (% MPE) of the drugs on sciatic nerve blockades at different doses. It can be seen from FIG. 8 that all the tested drugs produced dose-related sciatic nerve blockades of motor function, proprioception and nociceptive reaction. At a higher dosage, all the drugs produced 100% MPE on sciatic nerve blockade. Table 3 shows the ED₅₀ of the drugs on sciatic nerve blockades. It can be seen from Table 3 that on an ED₅₀ basis, the ranks of potencies of the drugs are: lidocaine hydrochloride>dextromethorphan hydrobromide monohydrate>3-methoxymorphinan hydrochloride>dextrorphan tartrate (p<0.01). In addition, dextromethorphan hydrobromide monohydrate and 3-methoxymorphinan hydrochloride had a more potent sciatic nerve blocking effect on nociceptive reaction than on motor function (p<0.01).

TABLE 3
ED₅₀ of drugs on sciatic nerve blockades of motor function, proprioception and
nociceptive reaction in rats

| | Motor function | Proprioception | Nociceptive reaction | Mean |
|---|---|---|---|---|
| Drug | ED₅₀ (95% CI) | ED₅₀ (95% CI) | ED₅₀ (95% CI) | ED₅₀ |
| Lidocaine hydrochloride (L) | 2.67 (2.55–2.81) | 2.57 (2.38–2.79) | 2.35 (2.14–2.57) | 2.53 |
| Dextromethorphan hydrobromide | 3.95 (3.79–4.12) | 3.73 (3.49–3.98) | 3.49 (3.23–3.78) | 3.72 |
| monohydrate (DM) | | | | |
| 3-methoxymorphinan hydrochloride | 7.40 (6.77–8.08) | 6.76 (6.12–7.47) | 5.89 (5.45–6.37) | 6.68 |
| (3MM) | | | | |
| Dextrorphan tartrate (DX) | 14.66 (13.78–15.58) | 14.52 (13.40–15.73) | 13.38 (12.37–14.47) | 14.19 |

Note 1:
95% CI refers to 95% confidence interval.
Note 2:
Difference among the drugs were compared using a one-way analysis of variance followed by pairwise Tukey honest significant difference test.

### 3. Duration:

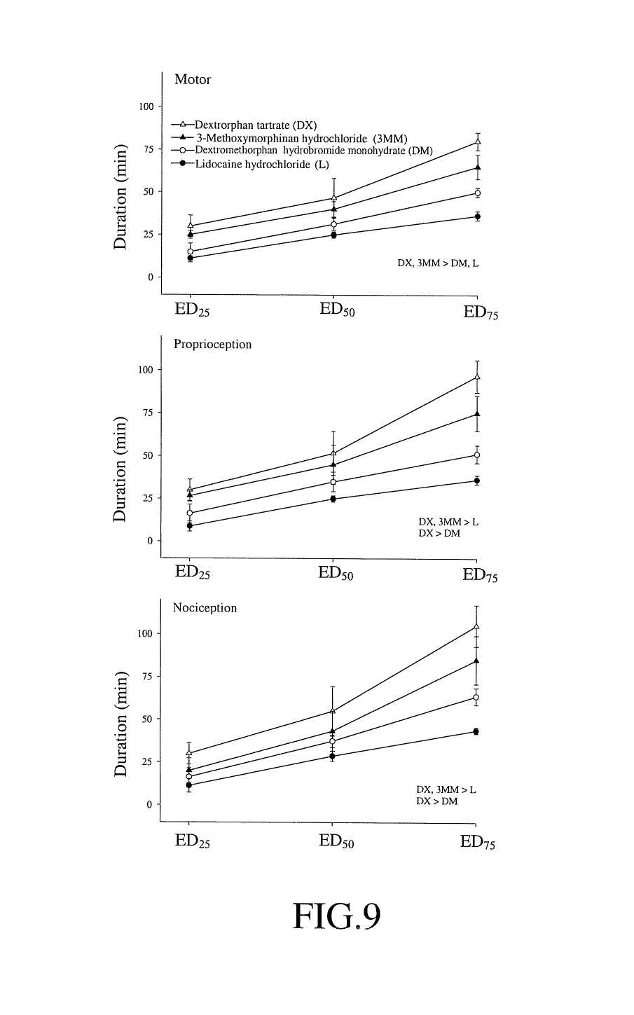

It can be seen from FIG. 9 that, at the same effective doses (i.e., ED₂₅ ED₅₀ or ED₇₅), dextromethorphan hydrobromide monohydrate, dextrorphan tartrate and 3-methoxymorphinan hydrochloride had a longer reaction duration than lidocaine hydrochloride.

### 4. Effect of Co-Administration:

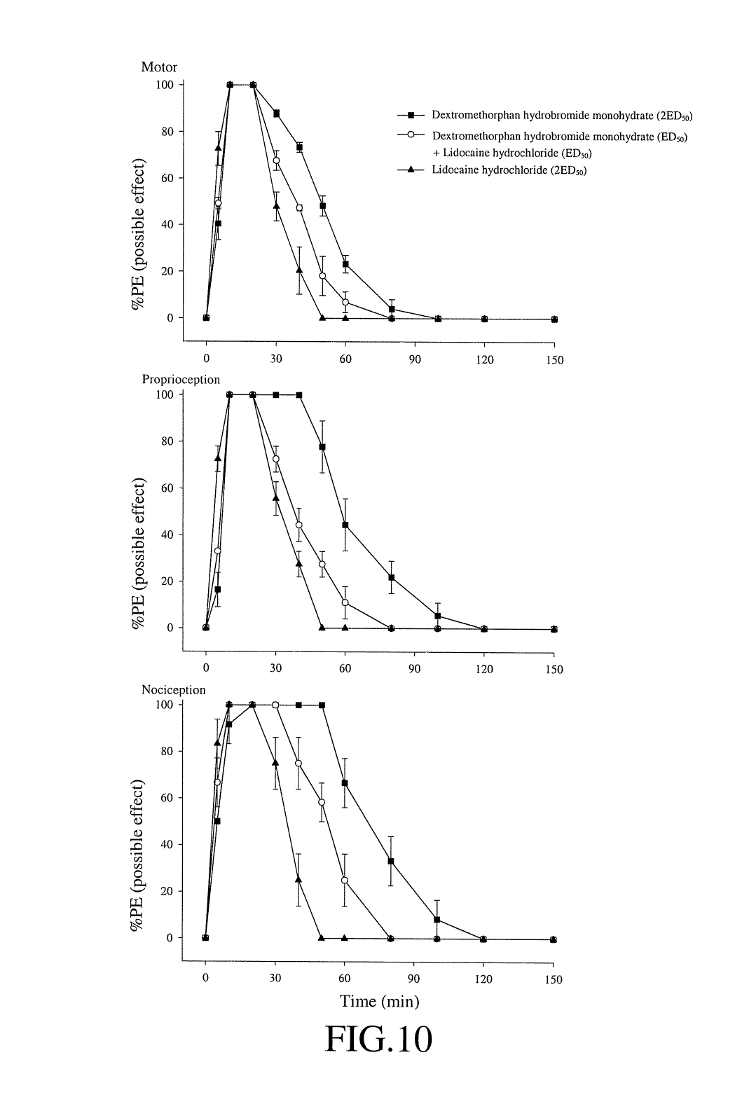

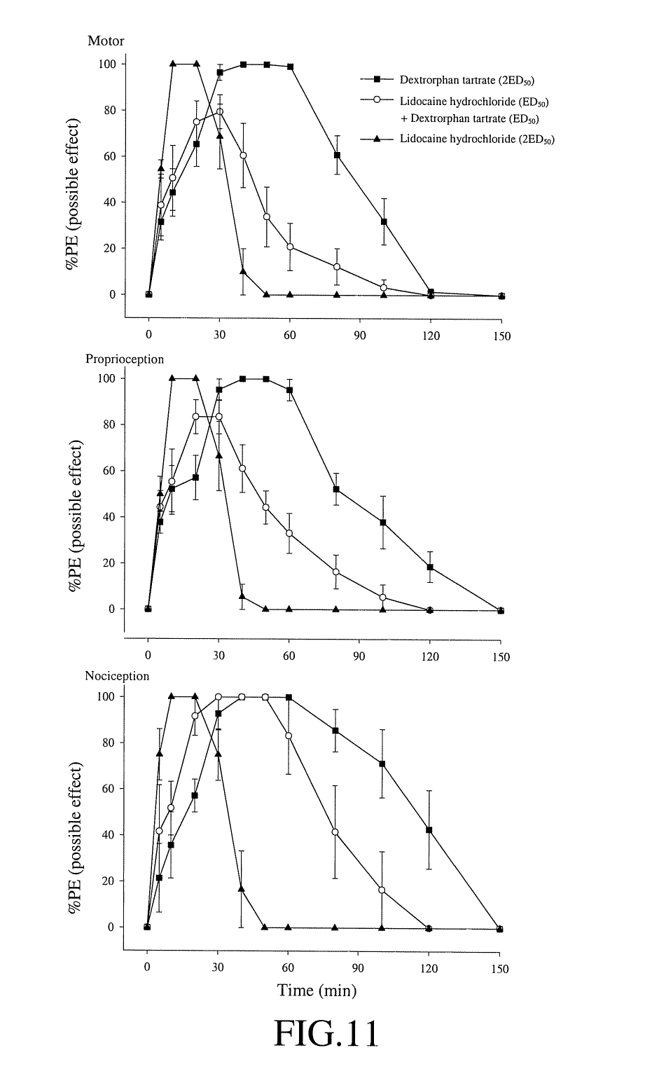

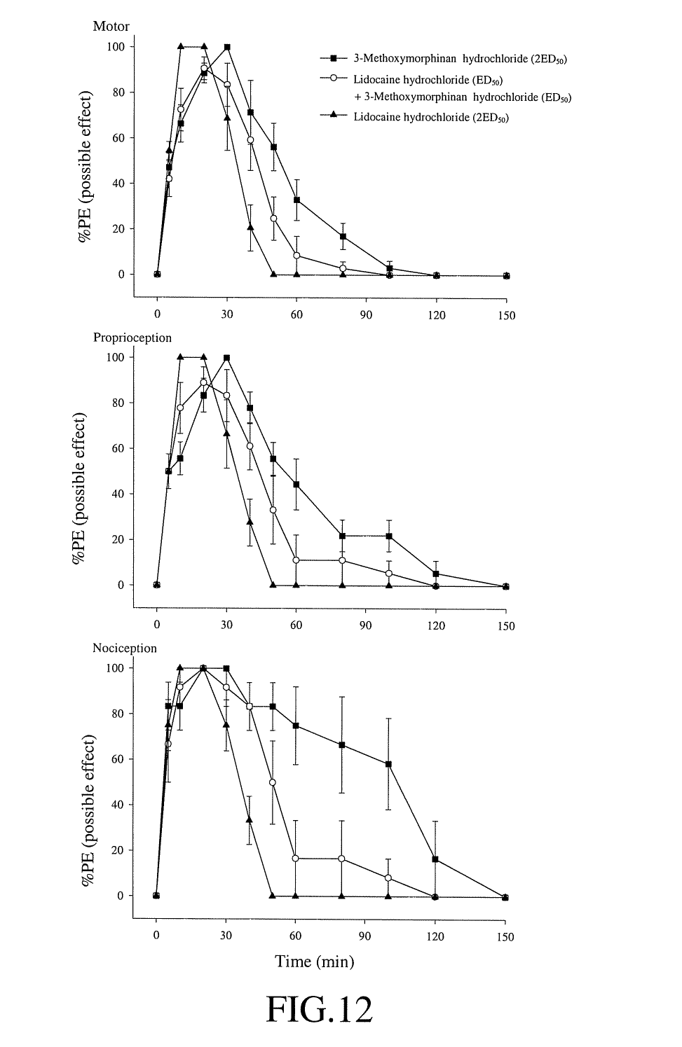

Effect of co-administration of dextromethorphan hydrochloride monohydrate, dextrorphan tartrate or 3-methoxymorphinan hydrochloride, with lidocaine on sciatic nerve blockade was evaluated. The results show that the AUC produced by the co-administration of dextromethorphan hydrobromide monohydrate and lidocaine hydrochloride on the sciatic nerve blockade of motor function, proprioception and nociceptive reaction was between the AUCs (see Table 4 and FIG. 10) produced when dextromethorphan hydrobromide monohydrate and lidocaine hydrochloride were administered independently. Similarly, co-administration of dextrorphan tartrate or 3-methoxymorphinan hydrochloride with lidocaine hydrochloride exhibited the same results as the co-administration of dextromethorphan hydrobromide monohydrate and lidocaine hydrochloride (see Table 4 and FIGS. 11-12).

TABLE 4
Effect of co-administration of dextromethorphan hydrobromide monohydrate,
dextrorphan tartrate or 3-methoxymorphinan hydrochloride with lidocaine
hydrochloride on sciatic nerve blockade in rats

| | Motor | | | | Nociceptive | |
|---|---|---|---|---|---|---|
| | function | | proprioception | | reaction | |
| Dextromethorphan | 4056 ± 212 | DM > L | 5498 ± 507 | DM > DM + L, | 6295 ± 643 | DM > DM + L, L |
| hydrobromide | | (p < 0.05) | | L (p < 0.05) | | (p < 0.05) |
| monohydrate (DM) | | | | | | |
| Dextromethorphan | 3268 ± 258 | | 3367 ± 237 | | 4479 ± 353 | |
| Hydrobromide | | | | | | |
| monohydrate + lidocaine | | | | | | |
| hydrochloride (DM + L) | | | | | | |
| Lidocaine hydrochloride (L) | 2594 ± 284 | | 2682 ± 204 | | 2886 ± 164 | |
| 3-methoxymorphinan | 4559 ± 584 | 3MM > L | 5191 ± 652 | 3MM > L | 8026 ± 1437 | 3MM > L |
| hydrochloride (3MM) | | (p < 0.05) | | (p < 0.05) | | (p < 0.05) |
| 3-methoxymorphinan | 3343 ± 414 | | 3794 ± 743 | | 4696 ± 959 | |
| hydrochloride + lidocaine | | | | | | |
| hydrochloride (3MM + L) | | | | | | |
| Lidocaine hydrochloride (L) | 2692 ± 248 | | 2808 ± 218 | | 2957 ± 253 | |
| Detroorphan tartrate (DX) | 7077 ± 429 | DX > DX + L, | 7357 ± 558 | DX > DX + L, L | 8786 ± 915 | DX > DX + L, L |
| | | L (p < 0.05) | | (p < 0.05) | | (p < 0.05) |
| Dextrorphan | 3448 ± 808 | | 3893 ± 470 | | 6177 ± 687 | DX + L > L |
| tartrate + lidocaine | | | | | | (p < 0.05) |
| hydrochloride (DX + L) | | | | | | |
| Lidocaine hydrochloride | 2505 ± 199 | | 2534 ± 196 | | 2694 ± 248 | |
| (L) | | | | | | |

Note 1:
Data were derived from AUC values in FIG. 10 to 12, and are expressed in mean ± SEM.
Note 2:
For each drug, the injection dosage was 2ED₅₀ (i.e., 2 times ED₅₀) when administered independently, the dosage was ED₅₀ when co-administered.
Note 3:
Differences among the drugs were compared using a one-way analysis of variance, followed by pairwise Tukey HSD test (p < 0.05).

The above results demonstrate that all of dextromethorphan hydrobromide monohydrate, dextrorphan tartrate and 3-methoxymorphinan hydrochloride have a sciatic nerve anesthetic effect, and have a longer duration than lidocaine hydrochloride. In addition, co-administration of dextromethorphan hydrobromide monohydrate, dextrorphan tartrate or 3-methoxymorphinan hydrochloride with lidocaine hydrochloride produced an additive effect on sciatic nerve blockades. Therefore, dextromethorphan hydrobromide monohydrate, dextrorphan tartrate and 3-methoxymorphinan hydrochloride are suitable for use as a local anesthetic to block peripheral nerves.

### Example 3

### Evaluation of Infiltrative Cutaneous Anesthetic Effect of dextromethorphan hydrobromide monohydrate, dextrorphan tartrate and lidocaine hydrochloride on Rats

### Experimental Animals:

Male Sprague-Dawley rats (weighing about 200 to 250 g); for each dose of each drug, n=8.

### Drugs:

Dextromethorphan hydrobromide monohydrate, dextrorphan tartrate, and lidocaine hydrochloride were freshly prepared in 0.9% saline. The injection doses of each drug were as follows:
(1) Dextromethorphan hydrobromide monohydrate: 0.49 mg/kg, 0.99 mg/kg, 1.45 mg/kg, 1.97 mg/kg, 2.47 mg/kg, 3.46 mg/kg, 4.94 mg/kg, and 6.91 mg/kg;
(2) Dextrorphan tartrate: 1.63 mg/kg, 2.72 mg/kg, 3.80 mg/kg, 4.56 mg/kg, 6.52 mg/kg, 8.15 mg/kg, 10.87 mg/kg, and 19.56 mg/kg; and
(3) Lidocaine hydrochloride: 1.81 mg/kg, 3.61 mg/kg, 5.41 mg/kg, 5.96 mg/kg, 7.22 mg/kg, 9.03 mg/kg, 10.83 mg/kg, and 18.05 mg/kg.

### Experimental Procedures:

### A. Drug Injection:

Regarding subcutaneous injection, reference was made to the method described by M. A. Khan et al. in Anesthesiology (2002), 96:109-116 and in Regional Anesthesia and Pain Medicine (2002), 27:173-179. In brief, hairs on the dorsal surface (about 10×10 cm²) of the thoracolumbar region of the rat were shaved, and the shaved surface was sterilized with iodine for preparation. To reduce the number of animals used, the back of the rat was further divided into left and right parts. One drug was injected first at the right part. After a washout period of one week, another drug was injected at the left part.

0.6 mL of drug was injected subcutaneously under the dorsal surface of the thoracolumbar region of the rat using a 30-gauge needle attached to a syringe (Becton Drive, Franklin Lakes, USA). The subcutaneous injection would result in a circular elevation (i.e., a wheal) of the skin, about 2 cm in diameter. The elevation was marked with ink within 1 minute after injection.

### B. Neurobehavioral Evaluations:

Effects of the drugs on infiltrative cutaneous anesthesia were evaluated by means of cutaneous trunci muscle reflex (CTMR) (see M. A. Khan et al. (2002), Anesthesiology, 96:109-116 and M. A. Khan et al. (2002), Regional Anesthesia and Pain Medicine, 27:173-179). CTMR refers to the reflex movement of the skin of the back caused by twitching of the lateral thoracispinal muscle in response to local dorsal cutaneous stimulation. Essentially, a von Frey filament (No.15; Somedic Sales AB, Sweden) to which a cut end of an 18-gauge needle was affixed was used to produce a standard nociceptive stimulus (strength: 19 g). Six pinpricks (at a frequency of 0.5-1 Hz) were applied to a single marked wheal during each test. The cutaneous anesthetic effect of the drugs was evaluated quantitatively as the number of times the pinpricks failed to elicit a response. For example, absence of any response to all of the 6 pinpricks was defined as a complete nociceptive block (i.e., 100% MPE). When three out of six pinpricks failed to elicit any response, this was defined as 50% MPE. When none of the six pinpricks failed to elicit a response, this was defined as 0% MPE. The six pinpricks were performed before injection and 2, 5, 10, 15, 20, 25, 30, 40, 50, 60, 75, 90, 115 and 120 minutes after injection until the CTMR completely recovered from the blockade.

### C. Potency Evaluation:

The potency evaluation was conducted with reference to the method described in Example 1.

### D. Evaluation of Duration:

The analysis of duration was conducted with reference to the method described in Example 1. The difference resided in that subcutaneous injections of ED₂₅, ED₅₀ and ED₇₅ of the drugs were performed based on the method described in the section “A. Drug injection” of this example, and evaluation of the infiltrative cutaneous anesthetic effect was performed based on the method described in the section “B. Neurobehavioral evaluation” of this example.

### E. Evaluation of Effect of Co-Administration:

The evaluation of the effect of co-administration was conducted with reference to the method described in Example 1. The differences resided in that lidocaine hydrochloride was used in place of bupivacaine hydrochloride, and that subcutaneous injection of the drugs and evaluation of infiltrative cutaneous anesthesia were conducted based on the methods described in the sections “A. Drug injection” and “B. Neurobehavioral evaluation” of this example.

### F. Evaluation of Systemic Safety Indices of Drugs:

The evaluation of the systemic safety indices of the drugs was conducted with reference to the method described by J. H. Kehne, et al., in Epilepsy Res. (1997), 27:41-54. The respective injection doses of each drug were as follows:
(1) Dextromethorphan hydrobromide monohydrate: 74.07 mg/kg, 111.10 mg/kg, 148.13 mg/kg, 222.20 mg/kg, and 240.71 mg/kg;
(2) Dextrorphan tartrate: 203.75 mg/kg, 224.13 mg/kg, 326.00 mg/kg, 407.50 mg/kg, and 611.25 mg/kg; and
(3) Lidocaine hydrochloride: 108.32 mg/kg, 162.48 mg/kg, 216.64 mg/kg, 240.71 mg/kg, and 270.80 mg/kg.

Essentially, each drug was injected intraperitoneally into the rats (n=8). The number of dead rats was observed after 24 hours, and the rate of mortality (%) was calculated. Thereafter, the doses of each drug and the relative mortality were plotted to obtain dose-mortality curves of the drugs. These curves were fitted by means of a computer-derived SAS Probit analysis (SAS Institute Inc., North Carolina), and the lethal doses (LD₅₀) of the drugs which would cause death of 50% of the rats were also calculated. The safety index (LD₅₀/ED₅₀) of each drug was calculated using the LD₅₀ thus obtained and the ED₅₀ obtained in the section “C. Potency evaluation” of this example.

G. Statistical analysis:

The statistical analysis was conducted with reference to the method described in Example 1.

### Result:

### 1. Time Course of Cutaneous Nociceptive Block at ED₇₅:

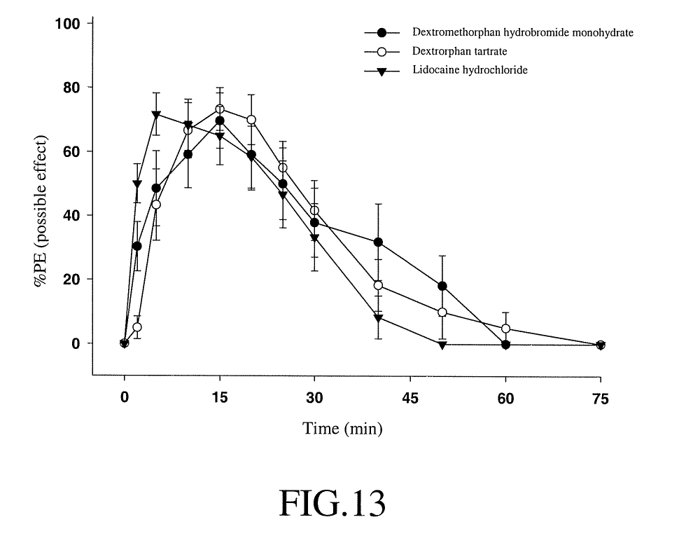

Due to the similarities of the graphs plotted for different doses, only the graph obtained at an injection dose of ED₇₅ is shown (see FIG. 13). It can be seen from FIG. 13 that, at ED₇₅, dextromethorphan hydrobromide monohydrate (ED₇₅ is 3.11 mg/kg), dextrorphan tartrate (ED₇₅ is 7.97 mg/kg), and lidocaine hydrochloride (ED₇₅ is 9.24 mg/kg) produced cutaneous nociceptive blocks of 71±8%, 73±7% and 72±7% MPE, respectively, and had durations of approximately 50±5, 48±3 and 33±4 minutes, respectively.

### 2. Potency Evaluation:

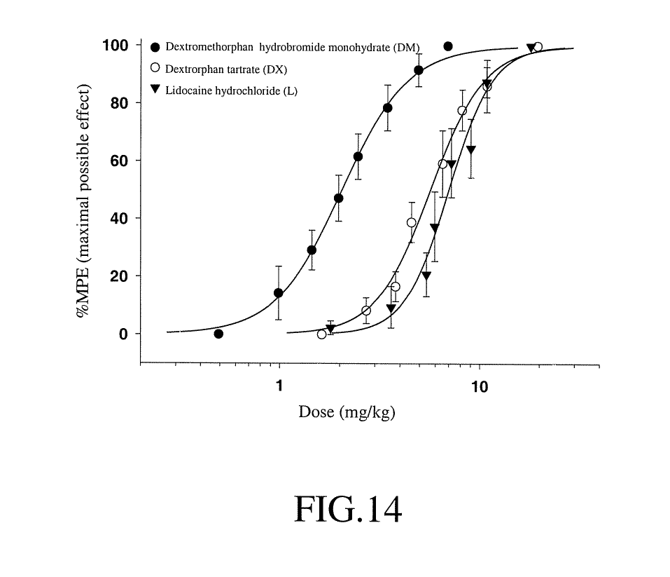

FIG. 14 shows the percentage of maximal possible effect (% MPE) on cutaneous nociceptive block at different doses of each drug. It can be seen from FIG. 14 that all the tested drugs produced a dose-related cutaneous anesthetic effect. Table 5 shows the ED, LD₅₀ and safety indices of the drugs. It can be observed from Table 5 that, on a basis of ED₅₀, the ranks of potency of the drugs are: dextromethorphan hydrobromide monohydrate>dextrorphan tartrate>lidocaine hydrochloride(p<0.01).

TABLE 5
Effective doses (ED), lethal doses (ED) and safety indices (LD₅₀/ED₅₀) of the
drugs on infiltrative cutaneous anesthesia in rats

| | ED | | | LD | |
|---|---|---|---|---|---|
| | ED₂₅ | ED₅₀ (95% CI) | ED₇₅ | LD₅₀ (95% CI) | LD₅₀/ED₅₀ |
| Dextromethorphan hydrobromide | 1.35 | 2.05 (1.84–2.29) | 3.11 | 138 (112–167) | 67.3 |
| monohydrate (DM) | | | | | |
| Dextrorphan tartrate (DX) | 4.03 | 5.66 (5.17–6.21) | 7.97 | 307 (257–369) | 54.3 |
| Lidocaine hydrochloride (L) | 5.31 | 7.00 (6.44–7.61) | 9.24 | 196 (161–222) | 28.1 |

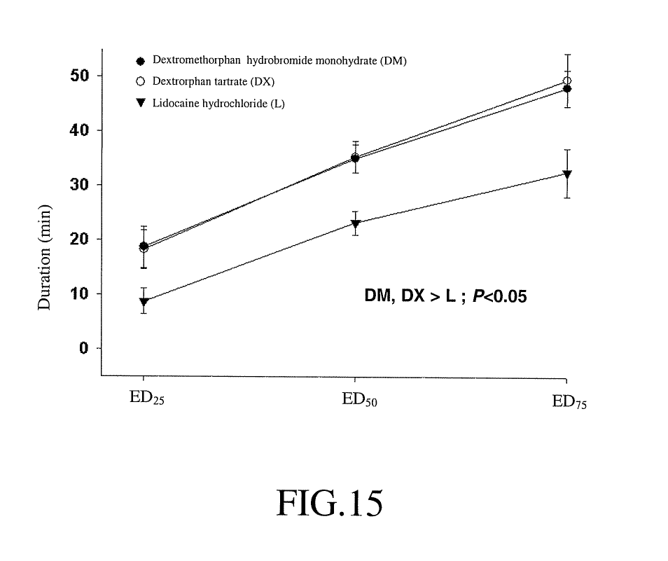

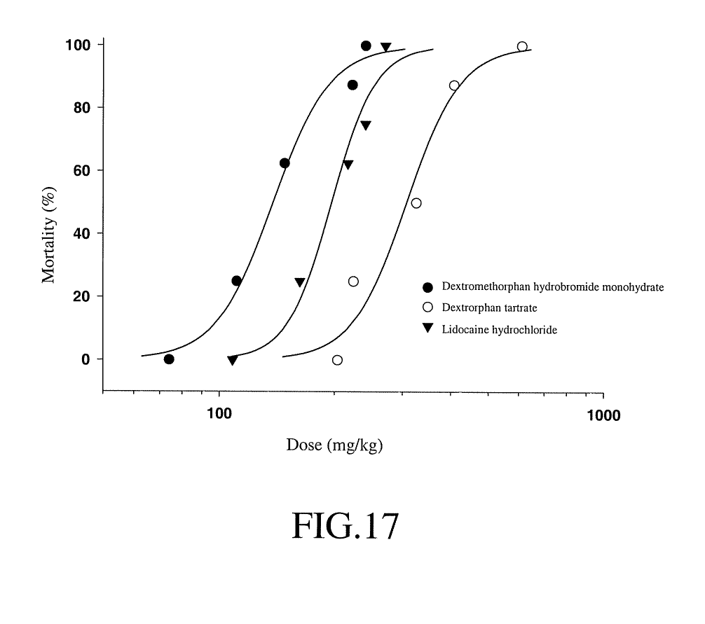

Note 1:
ED and LD data were derived respectively from FIGS. 15 and 17.
Note 2:
95% CI refers to 95% confidence interval.
Note 3:
Differences among the drugs were compared using a one-way analysis of variance followed by pairwise Tukey HSD test (p < 0.01).

### 3. Duration:

It is apparent from FIG. 15 that under the same effective doses (i.e., ED₂₅, ED₅₀ or ED₇₅), dextromethorphan hydrobromide monohydrate and dextrorphan tartrate had a longer duration than lidocaine hydrochloride.

### 4. Effect of Co-Administration:

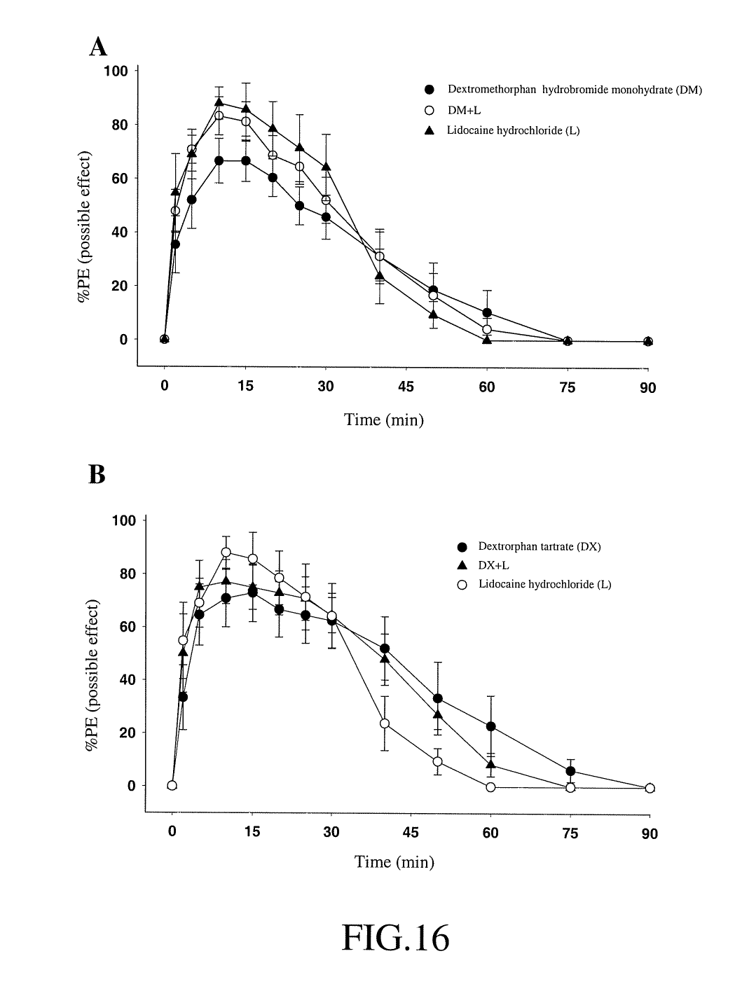

The effects of co-administration of dextromethorphan hydrobromide monohydrate or dextrorphan tartrate with lidocaine hydrochloride on cutaneous nociceptive block were evaluated. The results show that co-administration of dextromethorphan hydrobromide monohydrate and lidocaine hydrochloride and independent administration of dextromethorphan hydrobromide monohydrate or lidocaine hydrochloride did not have significant differences in terms of % MPE, duration and AUC (see Table 6 and FIGS. 16A). Similarly, co-administration of dextrorphan tartrate and lidocaine hydrochloride and independent administration of dextrorphan tartrate or lidocaine hydrochloride did not have significant differences in terms of % MPE, duration and AUC (see Table 6 and FIG. 16B). In addition, the results also show that co-administration of dextromethorphan hydrobromide monohydrate or dextrorphan tartrate with lidocaine hydrochloride produced an additive effect on cutaneous nociceptive blockade.

TABLE 6
Effects of co-administration of dextromethorphan hydrobromide
monohydrate or dextrorphan tartrate with lidocaine hydrochloride
on infiltrative cutaneous anesthesia of rats.

| | | Duration | AUC |
|---|---|---|---|
| | % MPE | (min.) | (% min.) |
| Dextromethorphan hydrobromide | 67 ± 8 | 58 ± 4 | 2300 ± 440 |
| monohydrate (DM) | | | |
| Dextromethorphan hydrobromide | 83 ± 7 | 52 ± 4 | 2500 ± 380 |
| monohydrate + lidocaine | | | |
| hydrochloride (DM + L) | | | |
| Lidocaine hydrochloride (L) | 90 ± 5 | 51 ± 3 | 2700 ± 360 |
| Dextrorphan tartrate (DX) | 73 ± 11 | 63 ± 7 | 3200 ± 690 |
| Dextrorphan tartrate + lidocaine | 77 ± 8 | 62 ± 3 | 3100 ± 390 |
| hydrochloride (DX + L) | | | |
| Lidocaine hydrochloride (L) | 88 ± 6 | 52 ± 4 | 2700 ± 410 |

Note 1:
Data are expressed in mean ± SEM.
Note 2:
AUC data were derived from AUC in FIG. 16.
Note 3:
The injection dosage of each drug when administered independently was 2ED₅₀ (i.e., 2 times ED₅₀); the injection dosage for co-administration was ED₅₀.

### 5. Drug Safety Evaluation:

The dose-mortality curve of each drug was constructed, and the LD₅₀ of each drug was obtained (see FIG. 17 and Table 5). In addition, the safety index (LD₅₀/ED₅₀) of each drug was also calculated (see Table 5). The results show that the systemic safety indices of dextromethorphan hydrobromide monohydrate and dextrorphan tartrate were 2.4 times and 1.9 times that of lidocaine hydrochloride, respectively. This indicates that dextromethorphan hydrobromide monohydrate and dextrorphan tartrate are suitable for use as an infiltrative cutaneous anesthetic.

The above results demonstrate that dextromethorphan hydrobromide monohydrate and dextrorphan tartrate are more effective in infiltrative cutaneous anesthetic effect than lidocaine hydrochloride, and have a longer duration.

All patents and literature references cited in the present specification are hereby incorporated by reference in their entirety. In case of conflict, the present description, including definitions, will prevail.

While the invention has been described with reference to the above specific embodiments, it is apparent that numerous modifications and variations can be made without departing from the scope and spirit of this invention. It is therefore intended that this invention be limited only as indicated by the appended claims.

We claim:

1. A method for inducing local anesthesia in a subject, comprising applying to the subject a dextrorotatory morphinan derivative of formula (I) or a pharmaceutically acceptable salt thereof: 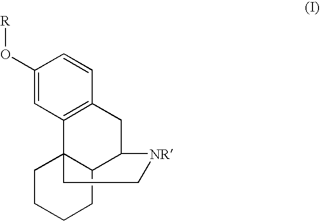
wherein R and R′ are independently selected from hydrogen and a methyl group, and at least one of R and R′ is a methyl group.

2. The method of claim 1, wherein the dextrorotatory morphinan derivative of formula (I) is dextromethorphan.

3. The method of claim 1, wherein the dextrorotatory morphinan derivative of formula (I) is 3-methoxymorphinan.

4. The method of claim 1, wherein the dextrorotatory morphinan derivative of formula (I) is dextrorphan.

5. The method of claim 1, wherein the dextrorotatory morphinan derivative of formula (I) or a pharmaceutically acceptable salt thereof is selected from group consisting of dextromethorphan hydrobromide monohydrate, dextrorphan tartrate, and 3-methoxymorphinan hydrochloride.

6. The method of claim 1, wherein applying the dextrorotatory morphinan derivative of formula (i) or a pharmaceutically acceptable salt to the subject is conducted via a parenteral route.

7. The method of claim 1, wherein applying the dextrorotatory morphinan derivative of formula (I) or a pharmaceutically acceptable salt to the subject is conducted via a route selected from epidural injection, intratracheal injection, subcutaneous injection, intrathecal injection, local transdermal delivery, local transmembrane delivery, injections that result in peripheral nerve plexus block, and injections that result in peripheral nerve block.

8. The method of claim 7, wherein applying the dextrorotatory morphinan derivative of formula (I) or a pharmaceutically acceptable salt to the subject is conducted via subcutaneous injection.

9. The method of claim 7, wherein applying the dextrorotatory morphinan derivative of formula (I) or a pharmaceutically acceptable salt to the subject is conducted via intrathecal injection.

10. The method of claim 7, wherein applying the dextrorotatory morphinan derivative of formula (I) or a pharmaceutically acceptable salt to the subject is conducted via an injection that results in peripheral nerve plexus block.

11. The method of claim 10, wherein the injection is a peri-sciatic nerve injection.

12. The method of claim 1, further comprising applying to the subject an additional local anesthetic.

13. The method of claim 12, wherein the additional local anesthetic is selected from the group consisting of bupivacaine, cocaine, mepivacaine, tetracaine, lidocaine, ropivacaine mesylate, their pharmaceutically acceptable salts, and combinations thereof.

14. The method of claim 13, wherein the additional local anesthetic is selected from the group consisting of bupivacaine, lidocaine, their pharmaceutically acceptable salts, and combinations thereof.

15. The method of claim 1, wherein the dextrorotatory morphinan derivative of formula (I) is formulated together with a pharmaceutically acceptable carrier.

16. The method of claim 15, wherein the pharmaceutically acceptable carrier is selected from the group consisting of water, saline, phosphate buffered saline, sugar-containing solutions, alcohol-containing aqueous solutions, oils, glycerin, organic solvents and liposomes.

17. The method of claim 16, wherein the pharmaceutically acceptable carrier is a 0.9% saline.

18. The method of claim 16, wherein the pharmaceutically acceptable carrier is a 5% dextrose solution.

19. The method of claim 1, wherein the dextrorotatory morphinan derivative of formula (I) is formulated further with an excipient selected from the group consisting of stabilizers, chelating agents, preservatives, emulsifiers, suspending agents, diluents, and gelling agents.
